SEQUENCE LISTING (18 sequences)

SEQ ID NO: 1 - DNA, 1350 bp, Artificial Sequence
  Synthesized
caggtgcagctccagcagccgggagcagaattggttaagcctggggcctcagtgaaaatgagctgtaaggccagcggctacaccttcacctcctataacatgcattgggtaaaacagacccccggcagaggtctcgagtggatcggagcgatttatccgggcaatggagacacttcctataatcagaaatttaagggcaaggccactctcacagccgacaagtcttcatccaccgcttatatgcagctgagctccttgacctctgaggacagcgccgtttactattgcgcacgaagcacgtactacgggggagattggtactttaacgtgtggggggccggaaccaccgtgactgtgtctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 450 aa, Artificial Sequence
  Synthesized
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 639 bp, Artificial Sequence
  Synthesized
cagattgtgttgtctcagtcccccgcaattctcagtgcgtcccccggcgaaaaggtgaccatgacctgccgcgcttcctcctcagtgagttatatccactggttccagcagaagccaggatcaagcccgaagccgtggatctacgccaccagcaacctggccagcggagtgcctgtgaggttctctggttctggcagcgggaccagttactcactcaccatttcccgggttgaggccgaagatgccgctacttattattgccaacagtggacctccaatccgccaacatttgggggagggactaaactggagattaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 4 - protein, 213 aa, Artificial Sequence
  Synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1347 bp, Artificial Sequence
  Synthesized
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 449 aa, Artificial Sequence
  Synthesized
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 639 bp, Artificial Sequence
  Synthesized
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 8 - protein, 213 aa, Artificial Sequence
  Synthesized
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 816 bp, Artificial Sequence
  Synthesized
cagattgtgttgtctcagtcccccgcaattctcagtgcgtcccccggcgaaaaggtgaccatgacctgccgcgcttcctcctcagtgagttatatccactggttccagcagaagccaggatcaagcccgaagccgtggatctacgccaccagcaacctggccagcggagtgcctgtgaggttctctggttctggcagcgggaccagttactcactcaccatttcccgggttgaggccgaagatgccgctacttattattgccaacagtggacctccaatccgccaacatttgggggagggactaaactggagattaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctatcgcaaatgtagaggaggccgaaggtggtgctaccaaaagggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 10 - protein, 272 aa, Artificial Sequence
  Synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSGGSGAKLAALKAKLAALKGGGGSYRKCRGGRRWCYQKGGGGSELAALEAELAALEAGGSGDSTYSLSSTLTLSKADYEKHKVYACEVTHQGLSSPVTKSFNRGEC

SEQ ID NO: 11 - DNA, 816 bp, Artificial Sequence
  Synthesized
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctatcgcaaatgtagaggaggccgaaggtggtgctaccaaaagggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 12 - protein, 272 aa, Artificial Sequence
  Synthesized
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSGGSGAKLAALKAKLAALKGGGGSYRKCRGGRRWCYQKGGGGSELAALEAELAALEAGGSGDSTYSLSSTLTLSKADYEKHKVYACEVTHQGLSSPVTKSFNRGEC

SEQ ID NO: 13 - DNA, 822 bp, Artificial Sequence
  Synthesized
cagattgtgttgtctcagtcccccgcaattctcagtgcgtcccccggcgaaaaggtgaccatgacctgccgcgcttcctcctcagtgagttatatccactggttccagcagaagccaggatcaagcccgaagccgtggatctacgccaccagcaacctggccagcggagtgcctgtgaggttctctggttctggcagcgggaccagttactcactcaccatttcccgggttgaggccgaagatgccgctacttattattgccaacagtggacctccaatccgccaacatttgggggagggactaaactggagattaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctgctatcgcaaatgtagaggaggccgaaggtggtgctaccaaaagtgtggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 14 - protein, 274 aa, Artificial Sequence
  Synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSGGSGAKLAALKAKLAALKGGGGSCYRKCRGGRRWCYQKCGGGGSELAALEAELAALEAGGSGDSTYSLSSTLTLSKADYEKHKVYACEVTHQGLSSPVTKSFNRGEC

SEQ ID NO: 15 - DNA, 822 bp, Artificial Sequence
  Synthesized
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctgctatcgcaaatgtagaggaggccgaaggtggtgctaccaaaagtgtggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 16 - protein, 274 aa, Artificial Sequence
  Synthesized
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSGGSGAKLAALKAKLAALKGGGGSCYRKCRGGRRWCYQKCGGGGSELAALEAELAALEAGGSGDSTYSLSSTLTLSKADYEKHKVYACEVTHQGLSSPVTKSFNRGEC

SEQ ID NO: 17 - DNA, 42 bp, Artificial Sequence
  Synthesized
tatcgcaaatgtagaggaggccgaaggtggtgctaccaaaag

SEQ ID NO: 18 - protein, 14 aa, Artificial Sequence
  Synthesized
YRKCRGGRRWCYQK

(19) United States
(12) Patent Application Publication
(10) Pub. No.: US 2022/0185903 A1
(43) Pub. Date: Jun. 16, 2022

(54) Novel bispecific binding molecule and drug conjugate thereof

(71) Applicant: NANTONG YICHEN BIOPHARMA. CO. LTD., Nantong (CN)

(72) Inventors: Feng WANG, Shanghai (CN); Huayang ZHENG, Shanghai (CN); Yuhan ZHANG, Shanghai (CN)

(21) Appl. No.: 17/436,092

(22) PCT Filed: Mar. 4, 2020
(86) PCT No.: PCT/CN2020/077793

§ 371 Date: Sep. 3, 2021

(30) Foreign Application Priority Data
Mar. 4, 2019 (CN) 201910160918.5

(51) Int. Cl. C07K 16/28; C07K 16/10; A61K 47/68; A61P 35/00; C07K 19/00

(52) U.S. Cl. CPC C07K 16/2887; C07K 16/1027; A61K 47/6849; A61K 47/6841; A61P 35/00; C07K 2317/92; C07K 2317/31; C07K 2317/77; C07K 2317/565; C07K 2317/24; C07K 2317/55; C07K 19/00

(57) ABSTRACT

Provided are a novel bispecific binding molecule and use thereof. The novel bispecific binding molecule-drug conjugate includes: a) a bispecific binding molecule consisting of a first binding moiety binds a tumor cell surface antigen (T) and a second antigen binding moiety binds to an internalizing effector protein (E), and b) a cytotoxic ingredient covalently conjugated to the bispecific binding molecule. The bispecific binding molecule may specifically exert cytotoxic activity against tumor cells by specifically targeting to the tumor cells through the first antigen binding moiety, and internalizing the cytotoxic ingredient into the tumor cells by the second antigen binding moiety of the bispecific binding molecule.

### CROSS-REFERENCE TO RELATED PATENT APPLICATIONS

The present application is a National Stage of International Patent Application No. PCT/CN2020/077793, filed Mar. 4, 2020, and claims the priority of Chinese Patent Application No. 201910160918.5, filed on Mar. 4, 2019, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy is named PN160531_SEQ LIST.txt and is 32 kilobytes in size. The Sequence listing contains 18 new sequences from SEQ ID NO:1 to SEQ ID NO:18, replaces the original sequence listing containing the sequences of SEQ ID NO: 1-SEQ ID NO:142 in the corresponding international application No. PCT/CN2020/077793, because the original sequence listing containing the sequences of SEQ ID NO: 1-SEQ ID NO:142 was wrongly filed on Mar. 4, 2020. The content of the replacement sequence listing is identical in substance to the sequences disclosed in the PCT application (especially in the example 1 of the present specification) and includes no new matter.

### TECHNICAL FIELD

The present invention relates to a novel bispecific binding molecule, a drug conjugate containing the bispecific binding molecule, and a pharmaceutical composition and use thereof.

### BACKGROUND

Antibody drug conjugates (ADCs) are a type of rapidly developed tumor-targeted therapeutic drugs. They use the exquisite specificity of monoclonal antibodies to tumor surface receptors to deliver and release cytotoxic drugs to tumor cells, greatly improving the therapeutic index (TI) of cytotoxic drugs. So far, two ADCs have been approved for marketing by the Food and Drug Administration (FDA), and more than 30 ADCs for hematological malignancies or solid tumors are in different stages of clinical development.

An ideal target receptor for ADCs should be highly expressed on the tumor surface, but rarely or not expressed in normal tissues to ensure the efficiency and accurate of loaded drugs delivered to tumor cells. In addition, ADCs have to be efficiently internalized by target cells and transported to lysosome to ensure drug release. Many tumor-associated antigens are not easily internalized or do not easily reach lysosomes, so the design of the novel ADCs is still limited by the lack of targetable tumor-specific receptors.

International application WO2017/134197 describes a multispecific antibody with internalizing properties that may simultaneously bind a target molecule and an internalizing effector protein. It promotes specific targeted killing of tumor cells by targeting the tumor cell surface antigen HER2.

CXCR4 (CD184) is a member of the G protein-coupled receptor family. Studies have found that CXCR4 is widely expressed on various tumors and is a poor prognostic marker for cancers such as breast cancer, colon cancer, melanoma and acute myelogenous leukemia. In addition, the expression of CXCR4 may be up-regulated in metastatic malignancies and cancer stem cells (CSC). A number of studies have evaluated the possibility of CXCR4 as a potential target for the treatment of hematological malignancies and metastatic solid tumors. At present, a plurality of CXCR4 antagonists, including AMD3100 (Plerixafor, a small molecule CXCR4 antagonist), BTK140 (a 14-resodie synthetic peptide), as well as anti-CXCR4 antibody (e.g. BMS-936564), are in the clinical trials for the treatment of acute myeloid leukemia (AML) and multiple myeloma. In addition, CXCR4 may be efficiently internalized by target cells upon its ligand binding. CXCR4-targeted ADC strategies are proposed as a potential treatment of various tumors. However, CXCR4 is widely expressed on normal cells, especially on hematopoietic cells (such as a T lymphocyte and a B lymphocyte), CXCR4-targeted ADCs may cause toxicity to normal tissues. B lymphocyte specific antigen CD20 is a well validated therapeutic target for the treatment of B cell-related malignancies. However, the treatment tolerance greatly limits the effectiveness of anti-CD20 antibodies. In addition, CD20 is poorly internalized after binding its receptor thereof, thus limiting its use as a tumor specific receptor for ADCs.

Therefore, the purpose of the present invention is to provide an ADC that specifically binds tumor stem cells, and has increased cytotoxicity to tumor stem cells, improved internalizing ability and reduced side effects.

### SUMMARY

The present application provides a novel bispecific binding molecule, which comprises: a) a first binding moiety that binds a tumor cell surface antigen (T) and a second binding moiety that binds an internalizing effector protein (E), wherein the first binding moiety is an antibody or an antigen binding fragment thereof, and the second binding part is a non-immunoglobulin polypeptide. In some embodiments, the second binding moiety is inserted into constant region of light chain of the first binding moiety through a connecting peptide; and in other embodiments, the second binding moiety is fused with C-terminal of constant region of the light chain of the first binding moiety.

The present application also provides a drug conjugate, which comprises the above bispecific binding molecule and a cytotoxic component covalently coupled to the bispecific binding molecule.

The inventors found that the bispecific binding molecule-drug conjugate of the present invention uses the first binding moiety that binds the cell surface antigen (T) as a carrier to deliver the bispecific binding molecule-drug conjugate to the surface of tumor cells. The cytotoxic drugs coupled to the bispecific binding molecule are effectively internalized into tumor cells by the second binding moiety which binds the internalizing effector protein, internalizing, thus greatly improved the therapeutic index.

The inventors also found that the bispecific binding molecule and the drug conjugates thereof of the present invention allow full utilization of tumor stem cell surface antigens that are usually not internalized or poorly internalized, thereby greatly increasing the potential of these antigens as targets of ADCs.

The inventor also found that the bispecific binding molecule and the drug conjugates thereof of the present invention can deliver, antibodies, or antibody fragments that internalizing are originally cannot internalized or poorly internalized into tumor stem cell, greatly improving the effectiveness of treatment, and reducing side effects.

The present invention discloses the following technical solutions:

1. A bispecific binding molecule, comprising:
i) a first binding moiety, which specifically binds tumor antigen (T); and
ii) a second binding moiety, which specifically binds internalizing effector protein (E),
herein the first binding moiety is an antibody or an antigen binding fragment thereof, and the second binding moiety is a non-immunoglobulin polypeptide.

2. The bispecific binding molecule of technical solution 1, wherein the second binding moiety is inserted into the constant region of the light chain or the heavy chain of the first binding moiety.

3. The bispecific binding molecule of technical solution 1, wherein E is a molecule that may be internalized into cells and expressed on the cell surface.

4. The bispecific binding molecule of technical solution 1, herein E is a protein with an internalizing effect on the surface of tumor cells.

5. The bispecific binding molecule of technical solution 4, wherein E is a protein with an internalizing effect on the surface of tumor stem cells.

6. The bispecific binding molecule of technical solution 1, wherein E is a soluble ligand that binds an internalizable receptor on the cell surface.

7. The bispecific binding molecule of technical solution 1, wherein E is selected from the following group consisting of: CXCR4, HER2, CD63, CD29, MHC-I, Kremen-1, Kremen-2, LRP5, LRP6, a transferrin receptor, a metabotropic glutamate receptor 5, LDLr, MAL, V-ATPase or ASGR.

8. The bispecific binding molecule of technical solution 7, wherein E is CXCR4.

9. The bispecific binding molecule of technical solution 8, wherein the second binding moiety comprises an amino acid sequence with at least 95%, 96%, 97%, 98%, or 99% identity with YRKCRGGRRWCYQK (SEQ ID NO: 18), or consists of such an amino acid sequence.

10. The bispecific binding molecule of any one of technical solutions 1-9, wherein T is a tumor stem cell surface antigen.

11. The bispecific binding molecule of technical solution 10, wherein T is selected from the following group consisting of: SSEA3, SSEA4, TRA-1-60, TRA-1-81, SSEA1, CD133, CD90 (Thy-1), CD326 (EpCAM), Cripto-1 (TDGF1), PODXL-1, ABCG2, CD24, CD49f (Integrin a6), Notch2, CD 146 (MCAM), CD117 (c-KIT), CD26 (DPP-4), CXCR4, CD34, CD271, CD13, CD56 (NCAM), CD105, LGR5, CD114 (CSF3R), CD54 (ICAM-1), CXCR1, CXCR2, TIM-3, CD55 (DAF), DLL4, CD20, CD96, CD29 (Integrin β1), CD9, CD166 (ALCAM), ABCB5, Notch3, and CD123 (IL-3R).

12. The bispecific binding molecule of technical solution 11, wherein T is CD20.

13. The bispecific binding molecule of any one of technical solutions 1-9, wherein T is a virus-induced tumor antigen.

14. The bispecific binding molecule of technical solution 13, wherein T is a virus-induced tumor antigen.

15. The bispecific binding molecule of any one of the preceding technical solutions, wherein the bispecific binding molecule binds T and E simultaneously.

16. The bispecific binding molecule of technical solution 15, wherein the bispecific binding molecule binds T and CXCR4 simultaneously.

17. The bispecific binding molecule of any one of technical solutions 1-12, wherein the bispecific binding molecule binds CD20 and CXCR4 simultaneously.

18. The bispecific binding molecule of any one of technical solutions 13-16, wherein the bispecific binding molecule binds RSV virus F protein and CXCR4 simultaneously.

19. The bispecific binding molecule of any one of the preceding technical solutions, wherein the first binding moiety is a chimeric antibody, a humanized antibody, a human antibody, or a recombinantly modified part of the antibodies thereof.

20. The bispecific binding molecule of any one of technical solutions 1-12, wherein the first binding moiety comprises HCDR1, HCDR2 and HCDR3 in the heavy chain amino acid sequence as shown in SEQ ID NO: 2, and LCDR1, LCDR2 and LCDR3 in the light chain amino acid sequence as shown in SEQ ID NO: 4.

21. The bispecific binding molecule of any one of technical solutions 1-12, wherein the molecule comprises an amino acid sequence as shown in SEQ ID NO: 2, and an amino acid sequence as shown in SEQ ID NO: 14; or the molecule comprises an amino acid sequence as shown in SEQ ID NO: 2, and an amino acid sequence as shown in SEQ ID NO: 10.

22. The bispecific binding molecule of any one of technical solutions 13-16, wherein the first binding moiety includes HCDR1, HCDR2 and HCDR3 in the heavy chain amino acid sequence as shown in SEQ ID NO: 6, and LCDR1, LCDR2 and LCDR3 in the light chain amino acid sequence as shown in SEQ ID NO: 8.

23. The bispecific binding molecule of technical solution 22, wherein the molecule comprises an amino acid sequence as shown in SEQ ID NO: 6, and an amino acid sequence as shown in SEQ ID NO: 12; or the molecule comprises an amino acid sequence as shown in SEQ ID NO: 6, and an amino acid sequence as shown in SEQ ID NO: 16.

24. A drug conjugate comprising the bispecific binding molecule of any one of the preceding technical solutions and a cytotoxic ingredient, the cytotoxic ingredient is selected from a drug, a toxin or a radioisotope, wherein the cytotoxic ingredient is conjugated with the first binding moiety or/and the second binding moiety.

25. The drug conjugate of technical solution 24, wherein the cytotoxic ingredient selected from maytansine, DM1, DM4, calicheamicin, pyrrolobenzodiazepine (PBD), duocarmycin (CAS NO. 130288), duostatin, duostatin-3, duostatin-5, rapamycin (CC-1065), alistatin, monomethylalistatin E (MMAE), monomethylalistatin F (MMAF), SN-38, doxorubicin, dolastatin, IGN-based toxin, a-manitin, or analogs, derivatives or prodrugs of any one of the above.

26. A nucleic acid encoding the bispecific binding molecule of any one of technical solutions 1-23.

27. An expression vector comprising the nucleic acid of technical solution 26.

28. A host cell comprising the expression vector of technical solution 27.

29. A pharmaceutical composition comprising the bispecific binding molecule of any one of technical solutions 1-23 or the drug conjugate of any one of technical solutions 24-25, and a pharmaceutically acceptable carrier.

30. Use of the bispecific binding molecule of any one of technical solutions 1-23 or the drug conjugate of any one of technical solutions 24-25 in preparing a medicine, wherein the medicine is used for the treatment of cancer.

31. Use of the bispecific binding molecule-drug conjugate of any one of technical solutions 1-17 in preparing a medicine, wherein the medicine is used for the treatment of cancer.

The present invention uses the first binding moiety targeting the tumor stem cell surface antigen (T) as the carrier to deliver the second binding moiety targeting the internalizing effector protein (E) to the cell surface. The cytotoxic ingredient coupled to the antibody is also effectively delivered into the cells by the internalizing effector protein, greatly improving its therapeutic index (TI).

The bispecific binding molecule and its ADCs thereof of the present invention could efficiently deliver the antibodies, antibody fragments and the antibody drug conjugates and the like for tumor stem cell surface antigen, which are originally not internalized or poorly internalized, to the interior of cells by internalizing effector proteins-binding polypeptides, antibodies, or antibody fragments, greatly improving the effectiveness of the treatment, effectively reducing side effects, and having a wide range of clinical application.

Further, since the internalizing effector proteins are often expressed on non-target cells such as normal cells, the bispecific binding molecule of the present invention uses the non-immunoglobulin polypeptides instead of antibody polypeptides to bind the internalizing effector protein, and inserts them into the constant region of the light chain n of the first binding moiety, preventing the off-target effect of the bispecific molecule, namely preventing the antibody molecule from binding the non-target cells that only express the internalizing effector protein, but not the tumor surface antigen, thereby improving the accuracy of drug delivery, and reducing or even avoiding the toxic side effects of the off-target effect.

### BRIEF DESCRIPTION OF THE DRAWINGS

FIG. 1 shows SDS-PAGE of SYN-AC, RTX and RTX-AC.

FIG. 2 shows an ADC formed by an antibody coupled to NHS-PEG4-MMAE.

FIG. 3A-C shows QTOF after deglycosylation modification by PNGase and DTT reduction of RTX, RTX-AC, SYN-AC and their corresponding ADCs (RTX-MMAE, RTX-AC-MMAE and SYN-AC-MMAE).

FIG. 4A-B shows flow cytometry results of RTX, RTX-AC and SYN-AC binding Jurkat cells and BJAB cells.

FIG. 5 shows the killing effect of Ramos cells by antibodies or ADCs for 72 h.

FIG. 6 shows the killing effect of ADCs on Ramous cells and Jurkat cells in a co-cultured Ramos/Jurkat system.

### DETAILED DESCRIPTION OF THE EMBODIMENTS

The present invention is described in detail herein by reference in combination with the following definitions and embodiments. The contents of all patents and disclosed documents mentioned herein, including all sequences disclosed in these patents and disclosure, are expressly incorporated herein by reference.

Antibody

The “antibody” of the present invention refers to an immunoglobulin molecule, a fragment of the immunoglobulin molecule, or a derivative of any one of them, which has the following features: specifically binding an antigen under typical physiological conditions, preferably binding two different antigens (for example, for a bispecific antibody) for a period of time to induce, promote, enhance and/or modulate the physiological response related to the binding of the antibody to the antigen.

The term “antibody” herein, unless otherwise specified or is clearly contradictory in the context, includes fragments of the antibodies, namely antigen-binding fragments which retain the ability to specifically bind an antigen. It has been indicated that the antigen-binding function of the antibodies may be achieved by the fragment of the full-length antibodies.

Bispecific Binding Molecule

The “bispecific binding molecule” of the present invention is a binding molecule with two binding specificities. The molecule comprises: a first binding moiety, which is an antibody or an antigen binding fragment thereof, specifically binding tumor cell surface antigen (T); and a second binding moiety, which is a non-immunoglobulin polypeptide, specifically binding internalizing effector protein (E) through non-antigen-antibody action. A method for producing the bispecific antibody is known in the art, and may be used to construct the multispecific antigen binding molecule of the present invention. In a preferred embodiment, the second binding moiety is inserted into the constant region of the light chain of the first binding moiety through a connecting peptide.

Tumor Antigen

As used herein, the term “tumor antigen” includes proteins or polypeptides that are preferentially expressed on the surface of tumor cells. As used in this context, the expression “preferentially expressed” means that the expression level of the antigen on the tumor cells is at least 10% (for example, 10%, 20%, 30%, 40%, 50%, 60%, 70%, 80%, 90%, 100%, 110%, 150%, 200%, 400% or higher) higher than the expression level of the antigen on non-tumor cells. In some embodiments, the target molecule is selected from the antigen that is preferentially expressed on the surface of the tumor cells (such as solid tumor or hematological tumor cells). In a preferred embodiment, the tumor cell is a tumor stem cell. It is well known in the art that the tumor stem cells are a subset of a small number of cells with self-renewal and multi-differentiation potential in tumor tissues. They are highly tumorigenic and are the source of tumor genesis, metastasis, drug resistance and recurrence.

Non-limiting examples of specific tumor antigens include, for example, EGFR, HER2, HER3, HER4, MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, VEGFR-1 (FLT1), VEGFR-2 (KDR/FIK-1), VEGFR-3, PDGF-RA, PDGF-RB, IGF-1R, IGF2B3, K-RAS, N-RAS, Bly-S(BAFF), BAFF-R, EpCAM, SAGE, XAGE-1b, BAGE, MAGE protein (such as MAGE-1, MAGE-2, MAGE-3, MAGE-4, MAGE-6, MAGE-9, MAGE-10, MAGE-12), GAGE-1, GAGE-2, GAGE-8, GAGE-3, GAGE-4, GAGE-5, GAGE-6, GAGE-7, XAGE-1b/GAGED2a, RAGE-1, RBAF600, CD2, CD3, CD19, CD-11α, CD16A, CD19, CD20, CD21, CD22, dipeptidyl-peptidase 4 (CD26), CD30, CD32B, CD33, CD38, CD40, CD45, CD52, CD70, CD80, CD60, CD62, CD72, CD79a, CD79B, SLAMF7 (CD139), CD123, Ly6D, Ly6E, Ly6K, gp100/Pmel17, EDAR, GFRA1 (GDNF-Ra1), MRP4, RET, STEAP1, STEAP2, TENB2, E16 (LAT1, SLC7A5), SLC35D3, MPF, SCL34A2, Sema 5b, PSCAhlg, ETBR, MSG783, FcRH1, FcRH2, NCA, MDP, IL20Ra, EphA2, EphA3, EphB2R, ASLG659, GEDA, CXCR5, P2X5, LY64, IRTA2, TMEF1, TMEM46, TMEM118, LGR5, GPR19, GPR172A, GPC3, CLL1, RNF43, KISS1R, ASPHD1, CXORF61, HAVCR1, epiregulin, amphiregulin, lipophilin, AIM-2, ALDH1A1, α-actinin-4, ARTC1, BING-4, CALCA, CASP-5, CASP-8, cdc27, CDK4, CDKN2A, CLPP, COA-1, CPSF, Cw6, RANKL, DEK-CAN, DKK1, EFTUD2, elongation factor 2, ENAH (hMena), ETV6-AML1, EZH2, FLT3-ITD, FN1, G250, MN, CAIX, GnTVf, GPNMB, HERV-K-MEL, hsp70-2, IDO1, IL13Ra2, intestinal carboxyl esterase, kallikrein 4, KIF20A, KK-LC-1, KM-HN-1, LAGE-1, LDLR-fucosyl transferase AS fusion protein, Lengsin, M-CSF, lactoglobulin-A, MART-1, Melan-A/MART-1, MART2, MCSP, mdm-2, ME-1, Meloe, MMP-2, MMP-7, mucin, MUM-1, MUM-2, MUM-3, myosin class I, NA88-A, PAP, neo-PAP, NFYC, NY-BR1, NY-BR62, NY-BR85, NY-ESO1, NY-ESO-1/LAGE-2, RAB38/NY-MEL-1, OA1, OGT, OS-9, p53, PAX3, PAX5, PBF, PML-RARa, PRAME, PRDX5, PSMA (FOLH1), PTPRK, RGS5, Rho, RhoC, RNF43, RU2AS, protein isolate 1, SIRT2, SNRPD1, SOX10, Sp17, SSX-2, SSX-4, survivin, SYT-SSX1 or -SSX2, TAG-1, TAG-2, telomerase, TGF-β, TGF-beta RII, TRAG-3, triose phosphate isomerase, TRP-2, TRP2-INT2, VEGF, WT1, TRPM4, CRIPTO, glycoprotein IIb/IIIa receptor, glycolipid GD2, GD3, folate receptor 1 (FOLR1), IFNγ, IFNα, β, ω receptor 1, TROP-2, Glyco-protein NMB, MMP9, GM3, mesothelin, fibronectin extra-domainB, endoglin, Rhesus D, plasma kallikrein, CS, thymic stromallymphopoietin, mucosal addressin cell adhesion molecule, nectin 4, NGcGM3, DLL3, DLL4, CLEC12A, KLB, FGFR1C, CEA, BCMA, p-cadherin, FAP, DR1, DR5, DR13, PLK, B7-H3, c-Met, gpA33, gp100/Pmel17, gp100, TRP-1/gp75, BCR-ABL, AFP, ALK, β-chain protein, BRCA1, BORIS, CA9, caspase-8, CDK4, CTLA4, cyclin-B1, cyclin D1, cyclin-A1, CYP1B1, Fra-1, GloboH, glypican-3, GM3, HLA/B-RAF, hTERT, LMP2, Mesothelin, ML-IAP, NA17, OX40, p15, PPLR, PCTA-1, PLAC1, PRLR, PRAME, SART-1, SART-3, TAG-72, TMPRSS2, Tn, tyrosinase and urine plaque protein-3, CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, CXCR6, androgen receptor (AR), calcitriol receptor (CR), estrogen receptor (ER), corticotropin releasing hormone receptor (CRHR), glucagon receptor (GCGR), gonadotropin receptor (FSHR, LHR), or melanocortin 1 receptor (MC1R, MSHR). In a particularly preferred implementation scheme, the tumor antigen on the surface of the tumor stem cells includes CXCR4.

As used herein, the term “tumor antigen” also includes viral antigens that may induce tumors. In some embodiments, the tumor-inducing viral antigen is selected from the F protein of the RSV virus.

Internalizing Effector Protein

The “internalizing effector protein” of the present invention refers to a protein that may be internalized into a cell or participate in or contribute to reverse gradient membrane transport in other ways. In some cases, the internalizing effector protein is a protein that undergoes transcytosis: namely, the protein is internalized on one side of the cell and transported to the other side of the cell (for example, from top to base). In some embodiments, the internalizing effector protein is a protein expressed on the surface of the cell. In any cases, the binding of the second binding moiety to the internalizing effector protein causes the internalization of the entire bispecific binding molecule and any molecules conjugated therewith into the cell. The internalizing effector proteins that are directly internalized into a cell includes membrane-associated molecules (for example, transmembrane proteins, and GPI-anchored proteins), which undergoes cell internalization and are preferably processed by intracellular degradative and/or recycling pathways. Specific non-limiting examples of the internalizing effector proteins that are directly internalized into the cell include, for example, CXCR4, HER2, CD29, CD63, MHC-I (for example, HLA-B27), Kremen-1, Kremen-2, LRPS, LRP6, LRP8, transferrin receptor, metabotropic glutamate receptor 5, LDL-receptor, LDL-related protein 1 receptor, ASGR1, ASGR2, amyloid precursor protein-like protein-2 (APLP2), apelin receptor (APLNR), MAL (myelin and lymphocyte protein, also known as VIP17), IGF2R, vacuolar H+ATPase, diphtheria toxin receptor, folate receptor, glutamate receptor, glutathione receptor, leptin receptor, scavenger receptor (for example, SCARA1-5, SCARB1-3, CD36) and the like.

Cytotoxic Component

The “cytotoxic component” of the present invention refers to a drug that inhibits or prevents cell function and/or causes cell death or destruction. Cytotoxic components include, but are not limited to, radioisotopes (for example, At²¹¹, I¹³¹, I¹²⁵, Y⁹⁰, Re₁₈₆, Re¹⁸⁸, Sm¹⁵³, Bi²¹², P³², Pb²¹² and a radioisotope of Lu); chemotherapeutic agents or drugs (for example, methotrexate, doxorubicin, vinca alkaloids (vincristine, vinblastine, and etoposide), doxorubicin, melphalan, mitomycin C, chlorambucil, daunorubicin or other inserts); a growth inhibitor; an enzyme and a fragment thereof, such as a nucleotide decomposing enzyme; an antibiotic; a toxin (such as a small molecule toxin or an enzymatically active toxin of bacterial, fungal, plant or animal origin, including a fragment and/or a variant thereof); and various anti-tumor or anti-cancer agents disclosed below.

The “chemotherapeutic agent” is a chemical compound that may be used to treat cancer regardless of the mechanism of action. Categories of the chemotherapeutic agent include, but are not limited to: an alkylating agent, an antimetabolite, a spinner inhibitor plant alkaloid, a cytotoxic/antitumor antibiotic, a topoisomerase inhibitor, an antibody, a photosensitizer and a kinase inhibitor. Examples of the chemotherapeutic agent include: anthracyclines, such as epirubicin or doxorubicin, cyclophosphamide, combinations of anthracyclines and cyclophosphamide (“AC”); taxanes, such as docetaxel or pacific paclitaxel, 5-FU (fluorouracil, 5-fluorouracil, CAS number 51-21-8), lapatinib, capecitabine, gemcitabine, PD-0325901 (CAS number 391210-10-9, Pfizer), cisplatin (cisdiamine dichloroplatinum (II), CAS number 15663-27-1), carboplatin (CAS number 41575-94-4), temozolomide (4-methyl-5-oxo-2,3,4,6,8-pentazabicyclo[4.3.0]non-2,7,9-triene-9-carboxamide, CAS number 85622-93-1), tamoxifen ((Z)-2-[4-(1,2-diphenylbut-1-enyl)phenoxy]-N,N-dimethyl-ethylamine).

More examples of the chemotherapeutic agent include: oxaliplatin, bortezomib, Sutent, letrozole, imatinib mesylate, XL-518 (MEK inhibitor, WO 2007/044515), ARRY-886 (Mek inhibitor), SF-1126 (PI3K inhibitor), BEZ-235 (PI3K inhibitor), XL-147 (PI3K inhibitor), PTK787/ZK 222584, fulvestrant, leucovorin (folinic acid), rapamycin, Lonafarnib, sorafenib, gefitinib, irinotecan, tipifarnib, ABRAXANE™ (non-hydrogenated castor oil), pacific paclitaxel albumin engineered nanoparticle preparations (American Pharmaceutical Partners, Schaumberg, II), vandetanib, chlorambucil, AG1478, AG1571 (SU5271; Sugen), sirolimus, pazopanib, camphorarmide, thiotepa and cyclophosphamide; alkyl sulfonate, such as busulfan, improsulfan, and piposulfan; aziridines, such as benzodepa, carboquone, meturedepa and uredepa; ethyleneimine and methyl melamine, including hexamethyl melamine, triethylenemelamine, trietbhlene phosphoramide, trietbhlene thiophosphoramide and trimethyl melamine; polyacetyl (especially bullatacin and bullatacinone); camptothecin (including synthetic analog topotecan); bryostatin; anemonin; CC-1065 (including its adozelesin, calezelesin and bizelexin synthetic analogues); cryptophycin (especially cryptophycin 1 and cryptophycin 8); dolastatin; duocarmycin (including synthetic analogues KW-2189 and CB1-TM1); eleutheroside; pancratistatin; coralinomycin; spongistatin; nitrogen mustards, such as chlorambucil, chlornaphazine, cyclophosphamide, estramustine, ifosfamide, methoxy mustard, mechlorethamine oxide hydrochloride, melphalan, novoembichin, phenesterine, prednimustine, trofosfamide, uracil mustard; nitrosoureas, such as carmustine, chlorozotocin, fotemustine, lomustine, nimustine and ranimustine; antibiotics, such as enediyne antibiotics (for example, Calicheamicin, Calicheamicin γ1I, Calicheamicin ωI1 (Angew Chem. Intl. Ed. Engl. (1994) 33: 183-186), dynemicin, dynemicin A; bisphosphonates, such as clodronate; esperamicin; and new carcinogen chromophores and related chromoprotein enediyne antibiotics chromophore), aclacinomycin, actinomycin, anthramycin, azaserine, bleomycin, actinomycin C, carubicin, carminomycin, carzinostatin, chromomycin, actinomycin D, daunorubicin, detorubicin, 6-diazo-5-oxo-L-norleucine, morpholinodoxorubicin, cyanomorpholinodoxorubicin, 2-pyrrololinyldoxorubicin and deoxydoxorubicin, epirubicin, esorubicin, idarubicin, marcellomycin such as mitomycin C, mycophenolic acid, nogalamycin, olivomycin, peplomycin, porfiromycin, puromycin, triiron adriamycin, rodorubicin, streptonigrin, streptozotocin, tuberculocidin, ubenimex, zinostatin, zorubicin; antimetabolites such as methotrexate and 5-fluorouracil (5-FU); folic acid analogues such as denopterin, methotrexate, and trimetrexate; purine analogues such as fludarabine, 6-mercaptopurine, thiamiprine, and thioguanine; pyrimidine analogues such as ancitabine, azacitidine, 6-azauridine, carmofur, arabinoside, dideoxyuridine, doxifluridine, enocitabine, and floxuridine; androgens, such as calusterone, dromostanolone propionate, epitiostanol, mepitiostane, and testolactone; anti-adrenal agents, such as aminoglutethimide, mitotane, and trilostane; folic acid supplements, such as leucovorin; aceglatone; aldophosamidoglycoside; aminolevulinic acid; eniluracil; amsacrine; bisantrene; edatrexate; defosfamide; demecolcine; diaziquone; eflornithine; elliptinium acetate; epothilone; etoglucid; gallium nitrate; hydroxyurea; lentinan; lonidamine; maytansinoids, such as maytansine and ansamitocin; mitoguazone; mitoxantrone; mopidamol; nitracrine; pentostatin; phenamet; pirarubicin; losoxantrone; podophyllic acid; 2-ethylhydrazide; procarbazine; polysaccharide complexes (JHSNatural Products, Eugene, Oreg.); razoxane; rhizomycin; sizofiran; spirogermanium; kwas tenuazonowy; triaziquone; 2,2′,2″-trichlorotriethylamine; trichothecenes (T-2 toxin, verrucarins A, bacillosporin A, and anguidin); urethane; vindesine; dacarbazine; mannitol mustard; dibromomannitol; dibromodulcitol; pipobroman; gacytosine; arabinoside (Ara-C); cyclophosphamide; thiotepa; 6-thioguanine; mercaptopurine; aminopterin; platinum analogues such as cisplatin and carboplatin; vinblastine; etoposide (VP-16); ifosfamide; mitoxantrone; vincristine; vinorelbine; novantrone; teniposide; edatrexate; daunomycin; aminopterin; ibandronate; CPT-11; topoisomerase inhibitor RFS2000; difluoromethylornithine (DMFO); retinoids, such as retinoic acid; and pharmaceutically acceptable salts, acids and derivatives of any one of the above.

Pharmaceutical Composition

The pharmaceutical composition as described herein is prepared by mixing the bispecific binding molecule of the present invention with a desired purity and one or more optional pharmaceutically acceptable carriers, which is in the form of a lyophilized formulation or an aqueous solution. The pharmaceutically acceptable carrier is generally non-toxic to the recipient at the dose and concentration used.

The bispecific binding molecules of the present invention may be used as single active ingredient, or administered in combination with, for example, an adjuvants or with other drugs such as immunosuppressive or immunomodulatory agents or other anti-inflammatory agents, for example, for the treatment or prevention of acute lymphoblastoid leukemia (ALL), acute medullary leukemia (AML), adrenal cortical cancer, anal cancer, appendix cancer, astrocytoma, basal cell carcinoma, brain tumor, cholangiocarcinoma, bladder cancer, bone cancer, breast cancer, bronchial tumor, Burkitt lymphoma, cancer of unknown primary origin, heart tumor, cervical cancer, chordoma, chronic lymphocytic leukemia (CLL), chronic myelogenous leukemia (CML), chronic myeloproliferative neoplasm, colon cancer, colorectal carcinoma, craniopharyngioma, skin T-cell lymphoma, ductal carcinoma, embryonal tumor, endometrial cancer, ependymoma, esophageal cancer, nasal cavity glioma, fibrous histiocytoma, Ewing's sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular carcinoma, histiocytosis, Hodgkin lymphoma, hypopharyngeal cancer, intraocular melanoma, islet cell tumor, Kaposi's sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, occult primary metastatic squamous neck cancer, midline tract cancer involving NUT gene, oral cancer, multiple endocrine neoplasia syndrome, multiple myeloma, granuloma fungoides, myelodysplastic syndrome, myelodysplastic/myelodysplastic neoplasms, nasal cavity and paranasal sinus cancer, nasopharyngeal cancer, neuroblastoma, non-Hodgkin's lymphoma, non-small cell lung cancer, oropharyngeal cancer, osteosarcoma, ovarian cancer, pancreatic cancer, papillomatosis, paraganglioma, parathyroid cancer, penile cancer, pharyngeal carcinoma, pheochromocytoma, pituitary tumor, pleuropulmonary blastoma, primary central nervous system lymphoma, prostate cancer, rectal cancer, renal cell carcinoma, renal pelvis and ureter cancer, retinoblastoma, rhabdomyomas, salivary glands cancer, Sezary syndrome, skin cancer, small cell lung cancer, small intestine cancer, soft tissue sarcoma, spinal cord tumor, stomach cancer, T cell lymphoma, teratoma, testicular cancer, throat cancer, thymoma and thymic cancer, thyroid cancer, urethral cancer, uterine cancer, vaginal cancer, vulvar cancer and Wilms tumor.

Sequence Variants

It may be understood by those skilled in the art that the bispecific molecule of the present invention and its encoding nucleic acid molecule also encompass variants of specific sequences given in the present application. Variants as used herein refer to a nucleic acid sequence or a peptide sequence that differs in sequence from a reference nucleic acid sequence or peptide sequence, respectively, but they retain basic biological properties of the reference molecule. A sequence change of a nucleic acid variant may not change an amino acid sequence of a peptide encoded by a reference nucleic acid, or may result in amino acid substitution, insertion, deletion, fusion and truncation. The sequence change of a peptide variant is usually limited or conservative, so that the sequences of the reference peptide and the variant are very similar, and are the same in multiple regions. The variant and the reference peptide may differ by one or more substitutions, insertions, and deletions in any combinations in terms of the amino acid sequence. The variant of the nucleic acid or the peptide may be naturally occurring, such as an allelic variant, or may be a variant that is not known naturally occurring. The non-naturally occurring variants of the nucleic acid and the peptide may be prepared by a mutagenesis technology or by direct synthesis. In various embodiments, the variant sequence and the reference sequence have at least 99%, at least 98%, at least 97%, at least 96%, at least 95%, at least 94%, at least 93%, at least 92%, at least 91%, at least 90%, at least 89%, at least 88%, at least 87%, at least 86%, and at least 85% of the identity.

### EXAMPLES

The examples are merely illustrative, but not intended to limit the present invention in any form.

### Example 1: Construction of Antibody Eukaryotic Expression Vector

1.1 Construction of Antibody Eukaryotic Expression Vector of Rituxan Antibody or Synagis Antibody

The heavy chain of Rituxan (anti-CD20) antibody Fab (RTX-FabH), the light chain of Rituxan antibody Fab (RTX-FabL), the heavy chain of Synagis (anti-RSV) antibody Fab (SYN-FabH), and the light chain of Synagis antibody Fab (SYN-FabL) (synthesized by IDT Company) are amplified by PCR with PfuUltra II DNA polymerase (Agilent Technologies, Inc., CA). The amplified RTX-FabH and SYN-FabH fragments are cloned into a pFuse-hIgG1-Fc vector (containing the following amino acid mutations: E233P/L234V/L235A/ΔG236+A327G/A330S/P331S, the vector mutation is completed in our laboratory) (InvivoGen, CA) by Gibson assembly kit (NEB, MA) to obtain pFuse-RTX HC and pFuse-SYN HC, respectively, and the amplified RTX-FabL and SYN-FabL are cloned into a pFuse vector without a hIgG1 -Fc fragment (InvivoGen, CA) to obtain pFuse-RTX LC and pFuse-SYN LC, respectively. All constructed vectors are verified by sequencing, and the nucleic acid and amino acid sequence of each construct are shown in Table 1.

1.2 Construction of RTX-FabL-AC and SYN-FabL-AC Eukaryotic Expression Vector

RTX-FabL-AC(CXCR4 antagonistic peptide was inserted into the constant region of RTX-FabL, synthesized by IDT company) and SYN-FabL-AC(CXCR4 antagonistic peptide were inserted into the constant region of SYN-FabL, synthesized by IDT company) were amplified by PCR using PfuUltra II DNA polymerase (Agilent Technologies, Inc., CA), respectively. The amplified RTX-FabL-AC and SYN-FabL-AC fragments were cloned into the pFuse vector (InvivoGen, CA) without the hIgG1-Fc fragment by Gibson assembly kit (NEB, MA) to obtain pFuse-RTX LC-AC and pFuse-SYN LC-AC, respectively. All constructed vectors were verified by sequencing. The nucleic acid and amino acid sequence of each construct thus obtained are shown in Table 1.

TABLE 1
Sequence name

| | | Nucleic acid | Amino acid |
|---|---|---|---|
| | | sequence | sequence |
| Construct | Polypeptide chain | Seq ID No: | Seq ID No: |
| RTX | RTX-HC | 1 | 2 |
| | RTX-LC | 3 | 4 |
| SYN | SYN-HC | 5 | 6 |
| | SYN-LC | 7 | 8 |
| RTX-AC | RTX-HC | 1 | 2 |
| | RTX LC-AC | 9 | 10 |
| RTX-ACd | RTX-HC | 1 | 2 |
| | RTX LC-ACd | 13 | 14 |
| SYN-AC | SYN-HC | 5 | 6 |
| | SYN LC-AC | 11 | 12 |
| SYN-ACd | SYN-HC | 5 | 6 |
| | SYN LC-ACd | 15 | 16 |
| | CXCR4 antagonist | 17 | 18 |
| | peptide | | |

### Example 2: Expression and Purification of Bispecific Binding Molecule

The heavy chain and light chain expression vectors constructed in example 1 are transiently transfected into FreeStyle HEK293 cells (ThermoFisher) (pFuse-RTX HC and pFuse-RTX LC, pFuse-RTX HC and pFuse-RTX LC-AC, or PFuse-SYN HC and pFuse-SYN LC-AC are co-transfected, and the amount of heavy chain plasmids and light chain plasmids during transfection is a molar ratio of 1:1): 28 ml of FreeStyle HEK 293 (3×10⁷ cells/ml) were seeded in a 125 ml shaking flask, the plasmids diluted with 1 ml of Opti-MEM (Invitrogen) were added to 1 ml Opti-MEM containing 60 μl of 293Fectin (Invitrogen). After incubated for 30 min at room temperature the plasmid-293 Fectin mixture was added to the cell suspension. Cells were cultured at 125 rpm, at 37° C. and 5% CO₂. Cell culture supernatant was collected at 48 h and 96 h after the transfection, respectively, and purified by Protein G Resin (Thermo Fisher Scientific, IL). Ion exchange chromatography was performed by GE AKTA. The chromatography column used is MonoS 5/50GL, and buffer used in the ion exchange chromatography were Buffer A: 20 mM NaOAc, pH=5 and Buffer B: 20 mM NaOAc, 1 M NaCl, pH=5. SDS-PAGE were applied following the chromatography.

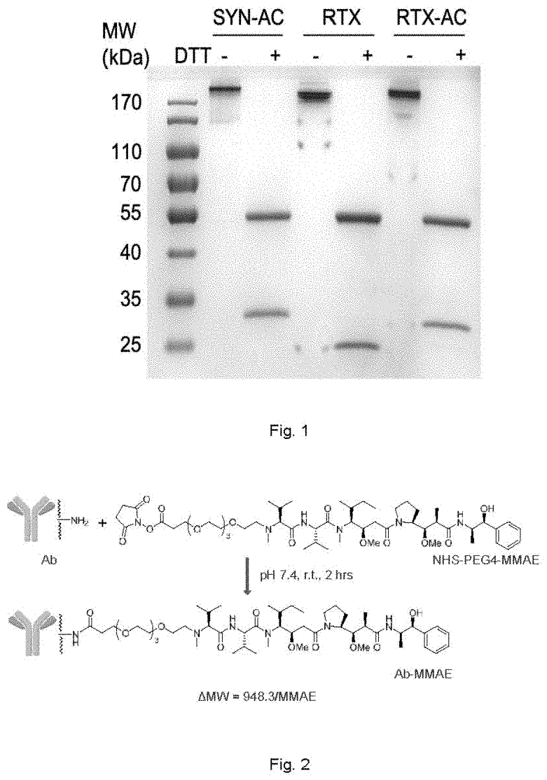

Results were shown in FIG. 1. Due to glycosylation modification, the molecular weight of RTX under non-reducing condition (about 170 kDa) is larger than the theoretical molecular weight (144 kDa), and the molecular weight of RTX-AC is slightly larger than that of RTX, indicating that the CXCR4 antagonist peptide may be successfully inserted. SYN-AC and RTX-AC have the similar migrations. Under reducing condition, the heavy chains of RTX, RTX-AC and SYN-AC have a band around 55 kDa (theoretical molecular weight 49 kDa) due to the glycosylation modification; the light chain of RTX appears at about 25 kDa, which is consistent with theoretical prediction. The light chains of RTX-AC and SYN-AC may have been inserted with the CXCR4 antagonist peptide, since their band positions were consistent with theoretical molecular weight.

### Example 3: Preparation of Bispecific Binding Molecule Drug Conjugate

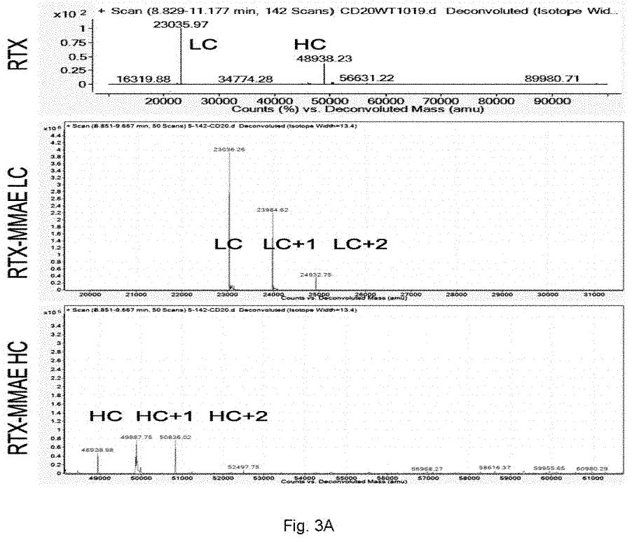

NHS-PEG4-MMAE (Concortis Biotherapeutics, USA) was synthesized. The purified RTX, RTX-AC and SYN-AC were buffered-exchanged with Amicon filter (EMD Millipore) into PBS buffer (500 μl with final concentration 5.8 μM, pH7.4), and followed by addition of 7.2 ul of NHS-PEG4-MMAE stock solution (10 mM, DMSO) (NHS-PEG4-MMAE, and the final concentration of NHS-PEG4-MMAE was 144 μM). The mixture was incubated for 2 h at room temperature, and purified by size exclusion chromatography (the chromatography column used is Superdex 200 Increase 101300 GL). A coupling process of the antibody and the drug was shown in FIG. 2.

### Example 4: Mass Spectrometry (MS) and Drug/Antibody Ratio (DAR) Analysis

RTX, RTX-AC, and SYN-AC or antibody drug conjugates RTX-MMAE-HC, RTX-MMAE-LC, RTX-AC-MMAE HC, RTX-AC-MMAE LC, SYN-AC-MMAE HC or SYN-AC-MMAE LC obtained in example 2 and 3 were incubated with PNGase F(NEB) at 37° C. for 8 hours. After treated with 10 mM of dithiothreitol, the sample was analyzed by ESI-Q-TOF-MS (Agilent, USA), and the drug/antibody ratio (DAR) was calculated according to a molecular weight measured by MS.

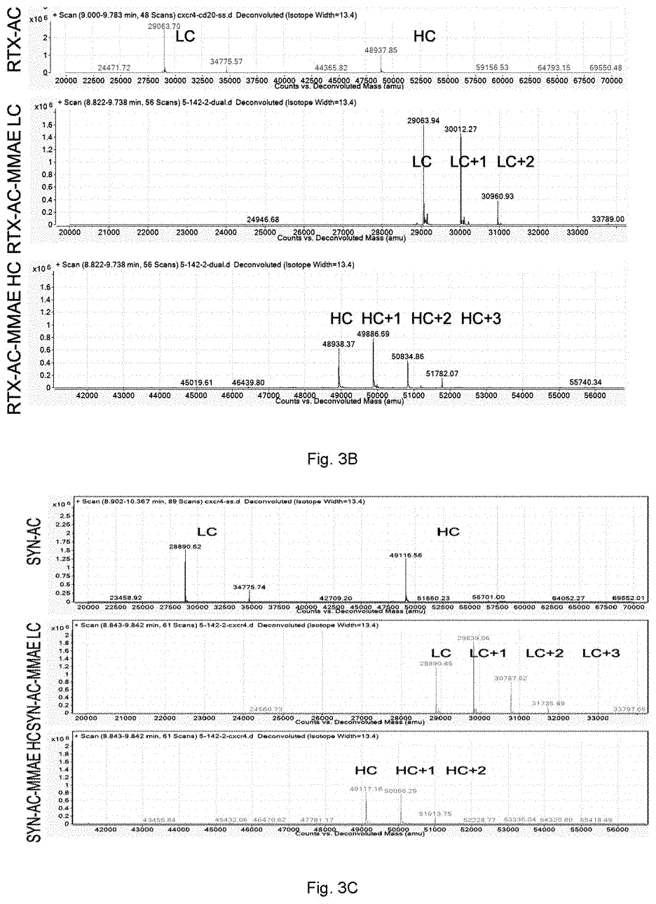

FIG. 3 showed QTOF mass spectrums of RTX, RTX-AC, SYN-AC and their corresponding ADCs (RTX-MMAE, RTX-AC-MMAE and SYN-AC-MMAE) after PNGase deglycosylation and DTT reduction. RTX-AC-MMAE had a peak at 48938 Da (similar to RTX-AC), and additional peaks at 49887 Da, 50834 Da, and 51788 Da, indicating that RTX-AC was conjugated with 1, 2 and 3 MMAEs, respectively. Similarly, a light chain mass spectrum of RTX-AC-MMAE also had two more peaks than that of the RTX-AC light chain, corresponding to the structure of 1 MMAE-conjugated and 2 MMAE conjugated RTX-AC light chain, respectively. These results indicated that the MMAE derivatives were successfully conjugated to RTX-AC. The drug/antibody ratios (DAR) of RTX-AC-MMAE, RTX-MMAE and SYN-AC-MMAE are 3.5, 3.2 and 3.3 as calculated from the ratio of the peaks seen in the mass spectrum, respectively.

TABLE 2
antibody and ADC molecular weight as detected by MS

| | Heavy chain | | | Light chain | | |
|---|---|---|---|---|---|---|
| Sample | MW (Da) | ΔMW (Da) | DAR | MW (Da) | ΔMW (Da) | DAR |
| RTX | 48938 | — | — | 23036 | — | — |
| | 48939 | 1 | 0 | 23036 | 0 | 0 |
| | 49888 | 950 | 1 | 23985 | 949 | 1 |
| RTX-MMAE | 50836 | 1898 | 2 | 24933 | 1897 | 2 |
| RTX-AC | 48938 | — | | 29064 | — | — |
| RTX-AC- | 48938 | 0 | 0 | 29064 | 0 | 0 |
| MMAE | 49887 | 949 | 1 | 30012 | 948 | 1 |
| | 50835 | 1897 | 2 | 30961 | 1897 | 2 |
| | 51782 | 2844 | 3 | | | |
| SYN-AC | 49117 | — | — | 28891 | — | — |
| | 49117 | 0 | 0 | 28891 | 0 | 0 |
| SYN-AC- | 50065 | 948 | 1 | 29839 | 948 | 1 |
| MMAE | 51014 | 1897 | 2 | 30788 | 1897 | 2 |

### Example 5: Analysis of In Vitro Activity of Bispecific Binding Molecule

Analysis of the Binding of the Bispecific Binding Molecule to CXCR4+/CD20−Jurkat Cells and CXCR4^{dim}/CD20⁺BJAB Cells by Flow Cytometry.

The CXCR4⁺/CD20⁻ Jurkat cells and CXCR4^{dim}/CD20⁺JAB cells were cultured in RPMI 1640 medium containing 10% FBS and 1%). Cells were collected by centrifugation, and washed with PBS for 3-4 times. After blocked by 1% BSA (in PBS) at 4° C. for 1 h, the cells were resuspended with PBS with 1% BSA at a density of 5×10⁵/100 μL. Different concentrations of RTX, RTX-AC or SYN-AC were added and gently mixed at 4° C. for 2 h. After being washed with PBS with 1% BSA for 3 times, cells were resuspended with PBS/1% BSA containing a PE-anti-human Fc antibody (Clone HP6017, Biolegend, CA), and incubated at 4° C. for 2 hours. Then an LSR II flow cytometer (Becton Dickinson, N.J.) was applied, and FlowJo software (TreeStar, OR) was used for analysis. Data was analyzed by nonlinear regression using a log (agonist) vs. response model in Prizm Graphpad software.

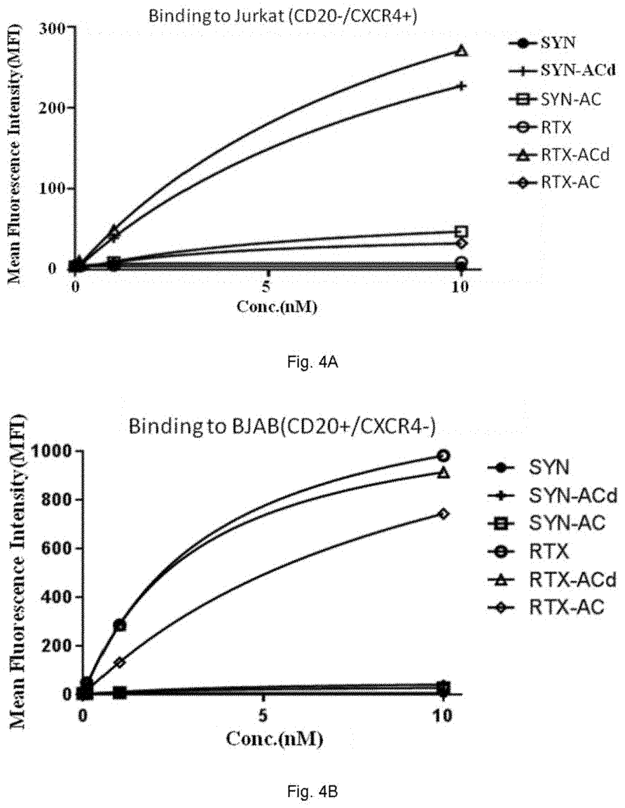

As shown in FIG. 4, RTX did not bind Jurkat cells. RTX-AC and SYN-AC has similar binding to Jurkat cells, which was dose-dependent. The binding of SYN-AC to BJAB cells was not strong, while the binding of RTX and RTX-AC to CD20+ BJAB cells was dose-dependent. The binding of RTX-AC to the BJAB cells was similar to that of RTX to BJAB, suggesting that the insertion of CXCR4 antagonist peptide into RTX had no effect on binding of RTX to CD20. In addition, RTX (via CD20), SYN-AC (via CXCR4) and RTX-AC (via CD20/CXCR4) may all bind to CXCR4⁺/CD20⁺ RAMOS cells.

### Example 6: Analysis of In Vitro Killing Activity of ADC

6.1 Killing Activity of ADC on CXCR4⁺/CD20⁺ Ramos Cells

Ramos cells (RPMI 1640 medium containing 10% FBS and 1% double antibody) were seeded in a flat-bottom 96-well plate (10⁴ cells/well, 90 ul/per well), and cultured overnight at 37° C. and 5% CO2. MMAE-PEG and protein solution were filtered through a 0.22 um filter membrane, gradiently diluted, and, added into the 96-well plate above (10 ul is added per well). 200 ul of solution is added to a gap between the wells to prevent evaporation. The 96-well plate was incubated at 37° C, 5% CO₂ for 72 hours. CellTiter Glo (Promega) was added to the wells, and fluorescence signals were detected by a plate reader (Molecular Devices). The data was processed and analyzed with GraphPad Prism software. The cell viability treated with PBS was defined as 100% (negative control) for normalization.

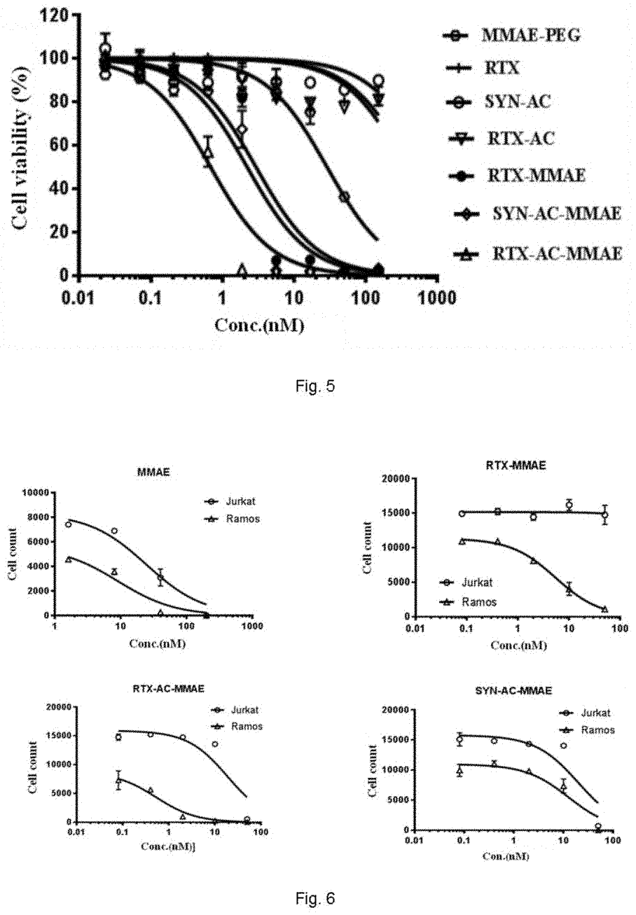

As shown in FIG. 5 and Table 3, ADCs (RTX-AC-MMAE, RTX-MMAE and SYN-AC-MMAE) were more effective in killing Ramos cells than the combination of unconjugated antibodies (RTX-AC, RTX, and SYN-AC) and MMAE-PEG. The killing effect of RTX-AC-MMAE on Ramos cells (EC₅₀=0.67 nM) was stronger than that of RTX-MMAE (EC₅₀=2.8 nM) or SYN-AC-MMAE (EC₅₀=2.3 nM).

TABLE 3
EC₅₀ of antibody or ADC on Ramos cells

| Sample | EC₅₀ |
|---|---|
| MMAE-PEG | 30.2 |
| RTX | >200 |
| SYN-AC | >200 |
| RTX-AC | >200 |
| RTX-MMAE | 2.8 |
| SYN-AC-MMAE | 2.3 |
| RTX-AC-MMAE | 0.67 |

6.2 Analysis of Killing Activity of ADC on Co-Cultured Ramos/Jurkat Cells

Ramos and Jurkat cells (RPMI 1640 medium containing 10% FBS and 1% double antibody) were cultured, respectively. Jurkat cells were stained with calcein-AM (eBioscience) and Ramos cells stained with Celltracker Organge CMTMR (Invitrogen) according to protocols provided by the manufacturer, followed by washed for three times with PBS. After cell count, Ramos and Jurkat cells were mixed at a ratio of 1:1, and 2×10⁴ Ramos/Jurkat mixed cells were added to a U-shaped bottom 96-well plate (90 ul/well). 10 ul of SYN-AC, RTX or RXT-AC with different concentrations was added to the 96-well plate above, and incubated at 37° C., 5% CO₂ for 72 hours. The numbers of the Ramos and Jurkat cells were counted with a flow cytometer (BD LSR II with a BD high throughput Sampler (HTS)).

Results were shown in FIG. 6 and Table 4. Compared with CD20−/CXCR4+ Jurkat cells, MMAE or SYN-AC-MMAE showed stronger killing activity on the CD20+/CXCR4+Ramos cells (IC₅₀ was shown in Table 3). The difference in cytotoxicity between MMAE and SYN-AC-MMAE may be due to the different sensitivity of these two cells to MMAE and the different expression levels of CXCR4 on the surface of these two cells. The killing effect of RTX-MMAE on Jurkat cells was weak, which was consistent with the fact that Jurkat cells lack CD20 expression. RTX-MMAE had a moderate cytotoxic effect on Ramos cells (EC₅₀=6.0 nM), which was related to weak CD20 internalization. RTX-AC-MMAE made full use of the characteristics and advantages of the two targets, IC₅₀ thereof was at a lower level (0.29 nM), and the selectivity thereof was better (therapeutic index, TI=69:1).

TABLE 4
EC₅₀ of ADC on co-cultured Jurkat and Ramos cells

| | EC₅₀ (nM) | | TI (therapeutic |
|---|---|---|---|
| Sample | Jurkat | Ramos | index) |
| MMAE | 24.5 ± 6.8 | 9.1 ± 3.8 | 2.7 |
| RTX-MMAE | N/A | 6.0 ± 1.2 | N/A |
| SYN-AC-MMAE | 21.0 ± 10.5 | 12.6 ± 1.4 | 1.7 |
| RTX-AC-MMAE | 20.1 ± 11.6 | 0.29 ± 0.17 | 69 |

What is claimed is:

1. A bispecific binding molecule, comprising:
i) a first binding moiety, wherein it specifically binds a tumor antigen (T); and
ii) a second binding moiety, wherein it specifically binds an internalizing effector protein (E),
wherein the first binding moiety is an antibody or an antigen binding fragment thereof, the second binding moiety is a non-immunoglobulin polypeptide;
wherein the second moiety is inserted into the constant region of light chain or heavy chain of the first binding moiety

2. (canceled)

3. The bispecific binding molecule according to claim 1, wherein E is selected from the followings: a molecule on a cell surface that can be internalized into the cell, a protein with an internalizing effect on the surface of tumor cells, a soluble ligand that bind an internalizing receptor on cell surface;
Wherein E is selected from the following group consisting of: CXCR4, HER2, CD63, CD29, MHC-I, Kremen-1, Kremen-2, LRP5, LRP6, a transferrin receptor, a metabotropic glutamate receptor 5, LDLr, MAL, V-ATPase or ASGR.

4. (canceled)

5. (canceled)

6. (canceled)

7. (canceled)

8. The bispecific binding molecule according to claim 3, wherein E is CXCR4.

9. The bispecific binding molecule according to claim 8, wherein the second binding moiety comprises an amino acid sequence having at least 95%, 96%, 97%, 98%, or 99% of the identity with YRKCRGGRRWCYQK (SEQ ID NO: 18), or consists of such an amino acid sequence.

10. The bispecific binding molecule according to any one claim 1, wherein T is a virus-induced tumor antigen or an antigen on the surface of tumor stem cell which selected from the following group consisting of: SSEA3, SSEA4, TRA-1-60, TRA-1-81, SSEA1, CD133, CD90 (Thy-1), CD326 (EpCAM), Cripto-1 (TDGF1), PODXL-1, ABCG2, CD24, CD49f (Integrin a6), Notch2, CD 146 (MCAM), CD117 (c-KIT), CD26 (DPP-4), CXCR4, CD34, CD271, CD13, CD56 (NCAM), CD105, LGR5, CD114 (CSF3R), CD54 (ICAM-1), CXCR1, CXCR2, TIM-3, CD55 (DAF), DLL4, CD20, CD96, CD29 (Integrin β1), CD9, CD166 (ALCAM), ABCB5, Notch3, and CD123 (IL-3R).

11. (canceled)

12. The bispecific binding molecule according to claim 10, wherein T is CD20 or RSV virus F protein.

13. (canceled)

14. (canceled)

15. (canceled)

16. (canceled)

17. The bispecific binding molecule according to claim 9, wherein the bispecific binding molecule simultaneously binds CD20 and CXCR4 or binds RSV virus F protein and CXCR4.

18. (canceled)

19. (canceled)

20. The bispecific binding molecule according to claim 9, wherein the first binding moiety comprises HCDR1, HCDR2 and HCDR3 in a heavy chain amino acid sequence as shown in SEQ ID NO: 2, and LCDR1, LCDR2 and LCDR3 in a light chain amino acid sequence as shown in SEQ ID NO: 4.

21. The bispecific binding molecule according to claim 9, wherein the molecule comprises an amino acid sequence as shown in SEQ ID NO: 2, and an amino acid sequence as shown in SEQ ID NO: 14.

22. The bispecific binding molecule according to claim 12, wherein the first binding moiety comprises HCDR1, HCDR2 and HCDR3 in a heavy chain amino acid sequence as shown in SEQ ID NO: 6, and LCDR1, LCDR2 and LCDR3 in a light chain amino acid sequence as shown in SEQ ID NO: 8.

23. The bispecific binding molecule according to claim 22, wherein the molecule comprises an amino acid sequence as shown in SEQ ID NO: 6, and an amino acid sequence as shown in SEQ ID NO: 12.

24. A drug conjugate comprising the bispecific binding molecule according to claim 9 and a cytotoxic ingredient, wherein the cytotoxic ingredient is selected from a drug, a toxin or a radioisotope, and the cytotoxic ingredient is conjugated with the first binding moiety or/and the second binding moiety.

25. The drug conjugate according to claim 24, wherein the cytotoxic ingredient is selected from maytansine, DM1, DM4, calicheamicin, pyrrolobenzodiazepine (PBD), duocarmycin (CAS NO. 130288), duostatin, duostatin-3, duostatin-5, rapamycin (CC-1065), alistatin, monomethylalistatin E (MMAE), monomethylalistatin F (MMAF), SN-38, doxorubicin, dolastatin, IGN-based toxin, a-manitin, or analogs, derivatives or prodrugs of any one thereof.

26. (canceled)

27. (canceled)

28. (canceled)

29. A pharmaceutical composition comprising the bispecific binding molecule according claim 1 and a pharmaceutically acceptable carrier.

30. (canceled)

31. The bispecific binding molecule according to claim 9, wherein the molecule comprises a heavy chain and a light chain, wherein the nucleic acids encoding the heavy chain and the light chain respectively are selected from any one of the following groups:
i) a heavy chain encoding nucleic acid sequence as shown in SEQ ID NO: 1 and a light chain encoding nucleic acid sequence as shown in SEQ ID NO: 9;
ii) a heavy chain encoding nucleic acid sequence as shown in SEQ ID NO: 1 and a light chain encoding nucleic acid sequence as shown in SEQ ID NO: 13;
iii) a heavy chain encoding nucleic acid sequence as shown in SEQ ID NO: 5 and a light chain encoding with nucleic acid sequence as shown in SEQ ID NO: 11;
iv) a heavy chain encoding nucleic acid sequence as shown in SEQ ID NO: 5 and a light chain encoding nucleic acid sequence as shown in SEQ ID NO: 15.

32. The bispecific binding molecule according to claim 9, wherein the molecule is isolated from a host cell transfected with an expression vector, wherein the expression vector comprising the nucleic acids.

33. A method for treatment of cancer, comprising: administrating therapeutically effective amount of the bispecific binding molecule according to claims 1 for a subject.

34. A method for treatment of cancer, comprising: administrating therapeutically effective amount of the drug conjugate according to claim 24 for a subject.

35. A pharmaceutical composition comprising the drug conjugate according to according claim 24 and a pharmaceutically acceptable carrier.

36. The pharmaceutical composition according to claim 35, wherein the cytotoxic ingredient is selected from maytansine, DM1, DM4, calicheamicin, pyrrolobenzodiazepine (PBD), duocarmycin (CAS NO. 130288), duostatin, duostatin-3, duostatin-5, rapamycin (CC-1065), alistatin, monomethylalistatin E (MMAE), monomethylalistatin F (MMAF), SN-38, doxorubicin, dolastatin, IGN-based toxin, a-manitin, or analogs, derivatives or prodrugs of any one thereof.
